(19) United States
(12) Patent Application Publication
(10) Pub. No.: US 2002/0055768 A1
(43) Pub. Date: May 9, 2002

(54) METHOD OF MANUFACTURING A THIN-LAYERED, ENDOVASCULAR, POLYMER-COVERED STENT DEVICE

(76) Inventors: KATHY HESS, MIDDLETON, MA (US); BARBARA KELLEY, BEDFORD, MA (US)

Correspondence Address: HOFFMAN & BARON LLP, 6900 JERICHO TURNPIKE, SYOSSET, NY, 11791

(21) Appl. No.: 09/448,702

(22) Filed: Nov. 24, 1999

(51) Int. Cl. A61F 2/06

(52) U.S. Cl. 623/1.13; 623/1.44; 623/1.49

(57) ABSTRACT

A stent-graft composite intraluminal prosthetic device comprises an elongated radially adjustable tubular stent and a polyolefin stent cover positioned about an exterior surface and/or interior surface thereof. The composite device is formed heat melting a film-like layer of polyolefin material onto a stent placed on a mandrel. The film has opposed longitudinal edges which are joined to form a tubular structure. The stent has a plurality of open spaces extending between opposed interior and exterior surfaces to permit radial adjustability, and the stent and cover are secured together through the open spaces of the stent. When both an exterior stent surface and interior stent surface are to be covered, such layers may be adheringly secured through the spaces by an adhesive, or laminated together through the open spaces of the stent.

### FIELD OF THE INVENTION

The present invention relates generally to a tubular implantable prosthesis including a stent and graft composite structure used to repair and/or replace or otherwise treat a body vessel. More particularly, the present invention relates to a stent-graft composite device including a radially deformable stent and a graft formed of a layer of polyolefin-based material wherein the layer covers at least an exterior surface of the stent.

### BACKGROUND OF THE INVENTION

Employment of various implantable tubular prostheses in medical applications is well known for the treatment of a wide array of vascular and other lumenal diseases. Such tubular prostheses are used extensively to repair, replace or otherwise hold open blocked or occluded body lumens such as those found in the human vasculature.

One type of prosthesis which is especially useful in maintaining the patency of a blocked or occluded vessel is commonly referred to as a stent. A stent is a generally longitudinal tubular device formed of biocompatible material which is useful in the treatment of stenosis, strictures or aneurysms in body vessels such as blood vessels. These devices are implanted within a vessel to reinforce collapsing, partially occluded, weakened or abnormally dilated sections of the vessel. Stents are typically employed after angioplasty of a blood vessel to prevent re-stenosis of the diseased vessel. While stents are most notably used in blood vessels, stents may also be implanted in other body vessels such as the urogenital tract and bile duct.

Stents are generally radially expandable tubular structures which are implanted intraluminally within the vessel and deployed at the occluded location. A common feature of stent construction is the inclusion of an elongate tubular configuration having open spaces therethrough which permit radial expansion of the stent. This configuration allows the stent to be flexibly inserted through curved vessels and further allows the stent to be radially compressed for intraluminal catheter implantation. Flexibility is a particularly desirable feature in stent construction as it allows the stent to conform to the bends in a vessel.

Once properly positioned adjacent the damaged vessel, the stent is radially expanded so as to support and reinforce the vessel. Radial expansion of the stent may be accomplished by inflation of a balloon attached to the catheter, or the stent may be of the self-expanding variety which will radially expand once deployed. Structures which have been used as intraluminal vascular stents have included coiled stainless steel springs; helically wound coil springs manufactured from a heat-sensitive material; and expanding stainless steel stents formed of stainless steel wire in a zig-zag pattern. Examples of various stent configurations are shown in U.S. Pat. Nos. 4,503,569 to Dotter; 4,733,665 to Palmaz; 4,856,561 to Hillstead; 4,580,568 to Gianturco; 4,732,152 to Wallsten and 4,886,062 to Wiktor.

Another implantable prosthesis which is commonly used in the vascular system is a vascular graft. Grafts are elongate tubular members typically used to repair, replace or support damaged portions of a diseased vessel. Grafts exhibit sufficient blood tightness to permit the graft to serve as a substitute conduit for the damaged vessel area. Grafts are also microporous so as to permit tissue ingrowth and cell endothelialization therealong. This improves the patency of the graft and promotes long term healing. Vascular grafts may be formed of various materials such as synthetic textile materials and fluoropolymers such as expanded polytetrafluoroethylene (ePTFE). Conventionally, graft materials have also been selected from polymers having high solubility factors, such as PET polyester and nylon.

If the graft is thin enough and has adequate flexibility, it may be collapsed and inserted into a body vessel at a location within the body having diameter smaller than that of the intended repair site. An intraluminal delivery device, such as a balloon catheter, is then used to position the graft within the body and expand the diameter of the graft therein to conform with the diameter of the vessel. In this manner, the graft provides a new blood contacting surface within the vessel lumen. An example of a graft device as discussed herein is provided in commonly assigned U.S. Pat. No. 5,800,512 to Lentz et al.

Composite stent-graft devices employing tubular structures are also known wherein a stent is provided with one or both of a polymeric cover disposed at least partially about the exterior surface of the stent and a polymeric liner disposed about the interior surface of the stent. These composite devices have the beneficial aspects of stents and grafts to hold open a blocked or occluded vessel and also replace or repair a damaged vessel thereby. Several types of stent-graft devices are known in the art. Examples of stent-graft composite devices are shown in U.S. Pat. No. 5,123,917 to Lee; U.S. Pat. No. 5,282,824 to Gianturco; U.S. Pat. No. 5,383,928 to Scott et al.; U.S. Pat. No. 5,389,106 to Tower; U.S. Pat. No. 5,624,411 to Tuch; and U.S. Pat. No. 5,674,241 to Bley et al.

While such composite devices are particularly beneficial due to the thinness at which they may be formed and the radial strength which they exhibit, the devices may suffer from a lack of biocompatibility in long-term applications, such as those in which therapeutic drugs are to be delivered over an extended period of time. The procedures which utilize all of the above disclosed devices obviate the need for major surgical intervention and reduce the risks associated with such procedures. However, none of the above described devices exhibit the biocompatibility required to significantly reduce tissue inflammation resulting from stent implantation and simultaneously extend the duration of implantation. Thus, it may be difficult to maintain an endovascular device having polymeric graft materials that induce inflammatory responses in native vessels.

Reduction of implantation-related inflammation can be effected by selection of graft materials that are inherently more biocompatible than those heretofore employed in stent-graft devices. Such materials include polyolefins, which are synthetic fibers made from an olefinic molecule that adds to itself, especially ethylene (giving polyethylene) or propylene (giving polypropylene). Polyolefinic materials have the useful property of being thermoplastic, softening at about 150° C. at which temperature they can be readily molded or extruded.

Polymer solubility is of considerable importance because the degree of decomposition of a polymeric material within the vascular system contributes to the extent of inflammation encountered with implantation of a prosthesis. Solubility of polymers is the extent to which polymers pass into solution. Solubilization may be very slow owing to the time needed for large chain molecules to diffuse into the fluid. Polyolefins are usually difficult to dissolve in any solvent at ambient temperature, so that high temperatures (160° C.) are needed to effect solubilization. This characteristic is desirable in the use of implantable tubular prostheses, for reduced solubility translates in reduced introduction of foreign matter into native vessels owing to decomposition of the polymeric materials. Higher solubility factors used in the fabrication of current prostheses using PET polyester and similar materials indicate that the material is prone to higher rates of solubilization within native vessels and therefore more prone to inflammatory responses. Such responses can translate in swelling of the surrounding vessel and impeded bloodflow therethrough as a result thereof. Inflammations can further lead to tissue ingrowth at the periphery of the prosthesis, further impeding bloodflow and defeating the purpose of the stent-graft device to not only maintain the patency of the vessel, but also assist in the healing of surrounding tissue.

The melting temperatures of olefinic polymers are relatively high. The high melting polymers are of considerable interest because relatively few thermoplastic polymers are available which have high softening temperatures and at the same time can be easily fabricated. Polyolefinic materials are also of interest because their solubility factors (7.9-8.1) give the material a more “bio-friendly” reaction with a native vessel

Broadly, polyolefin resins may include virtually all addition polymers; however the term polyolefin is specifically used for polymers of ethylene, the alkyl derivatives of ethylene and the dienes. Polyethylene is a whitish, translucent thermoplastic polymer of moderate strength and high toughness. The physical properties vary markedly with the degree of crystallinity and with the size and distribution of crystalline regions. With increasing crystallinity or density, polyethylene products generally become stiffer and stronger, and they acquire higher softening temperatures and higher resistance to penetration by liquids and gases. Polyethylene is a good insulator, easily molded and blown and highly resistant to acids. Polyethylene is often used to make films and sheets

High molecular weight polypropylene is generally similar in properties to high-density polyethylene. In comparison with the latter, isotactic polypropylene is harder and stronger. The melting temperature of polypropylene is high, and the density of polypropylene is the lowest of all solid polymers. Like polyethylene, polypropylene is often used in fibers to forms sheets and films.

Accordingly, it is desirable to implement a polyolefinic material in a stent-graft device which exhibits sufficient radial strength to permit the composite device to accommodate a radially expandable stent and yet improves biocompatibility with a vascular site into which implantation occurs. It is further desirable to provide an expandable tubular stent which exhibits sufficient radial strength to permit the stent to maintain patency in an occluded vessel and yet prevent reoccurrence of occlusions in a passageway by providing an expandable tubular stent of generally open, cylindrical configuration that utilizes polyolefin material having low solubility factors. Such a device prevents inflammation of lumen passageways due to incompatibility with graft material and assists in the healing of diseased lumen tissue by enabling extended elution of therapeutic substances therefrom.

### SUMMARY OF THE INVENTION

It is an advantage of the present invention to provide an improved tubular stent-graft composite device.

It is another advantage of the present invention to provide an easily manufactured stent-graft device which reduces tissue inflammation due to implantation of the device within vascular tissue.

It is a further advantage of the present invention to provide a stent-graft composite device having the dual function of structural support for a radially expandable stent and absorption and release of therapeutic agents.

The present invention provides a stent-graft composite intraluminal prosthetic device comprising an elongate radially adjustable tubular stent, defining opposed interior and exterior stent surfaces and a polymeric stent sheath covering at least the exterior surface of the stent. The stent can include a plurality of open spaces extending between the opposed exterior and interior surfaces so as to permit said radial adjustability. The stent has a polymeric material on its exterior surface, its interior surface, in interstitial relationship with the stent or any combination of the above. The polymer is preferably selected from the group of polymeric materials consisting of polyolefins, such as polyethylene and polypropylene, and preferably having melting temperatures in the range 300-400° C., inclusive. If separate sheaths are placed on both the exterior and interior surfaces of the stent, the sheaths are secured to one another through said open spaces, such as by lamination or adhesion using a thermoplastic adhesive such as fluorinated ethylene propylene (FEP).

A method of making a stent-graft endovascular prosthesis of the present invention is also disclosed. The disclosed method includes providing an elongated radially adjustable tubular stent, defining opposed interior and exterior stent surfaces. The stent is placed about a correspondingly sized and shaped mandrel and covered with a polymeric material on at least an exterior surface thereof. The covered stent is then heated to 300-400° C. for a time sufficient to melt said material over said stent. The covered stent is finally removed from the mandrel. When both an exterior stent and interior stent surface are to be covered, the polyolefin film may be affixed on the mandrel prior to affixing the stent thereon. The film layers can then be secured through the open spaces of the stent as described hereinabove.

### BRIEF DESCRIPTION OF THE DRAWINGS

FIG. 1 is a perspective view of a preferred embodiment of a tubular stent-graft prosthesis of the present invention.

FIG. 2 is a perspective view of one embodiment of a stent which may be used in a stent-graft composite prosthesis of the present invention.

FIG. 3 shows a schematic of a stent on a mandrel during fabrication of a tubular stent-graft prosthesis of the present invention.

FIG. 4 shows a schematic of the stent of FIG. 3 having a polyolefin film covering an exterior surface thereof and a heat shrink tubing thereover.

FIG. 5 shows a schematic of a tubular stent-graft prosthesis of FIG. 4 after removal from the mandrel.

FIG. 6 shows a cross-section of a preferred embodiment of the tubular stent-graft prosthesis of the present invention taken along line 6-6 of FIG. 5.

FIG. 7 shows a schematic of a polyolefin film on a mandrel prior to affixing a stent thereon.

FIG. 8 shows a schematic of the film and mandrel of FIG. 7 after placement of a stent thereover.

FIG. 9 shows a cross-section of a preferred embodiment of a tubular stent-graft prosthesis of the present invention having a polyolefin layer disposed about a luminal surface thereof after removal from the mandrel as taken along line 9-9 shown in FIG. 8.

FIG. 10 is a cross section of another embodiment of a tubular stent-graft prosthesis of the present invention having a stent with polyolefin layers disposed about both a luminal surface and an exterior surface thereof.

### DETAILED DESCRIPTION OF THE PREFERRED EMBODIMENTS

In the present invention, a tubular stent-graft prosthesis is provided which incorporates a tubular radially adjustable stent having a covering over an exterior and/or interior surface thereof. The covering is formed from a fiber of polyolefinic film, such as polyethylene or polypropylene, which is readily extruded at its softening temperatures, possesses high strength and softness and further exhibits low solubility characteristics which avoid tissue inflammatory responses. Polyolefins are utilized in combination with endovascular stent devices so as to decrease the inflammatory reactions in blood vessels that have heretofore been encountered with conventional graft materials.

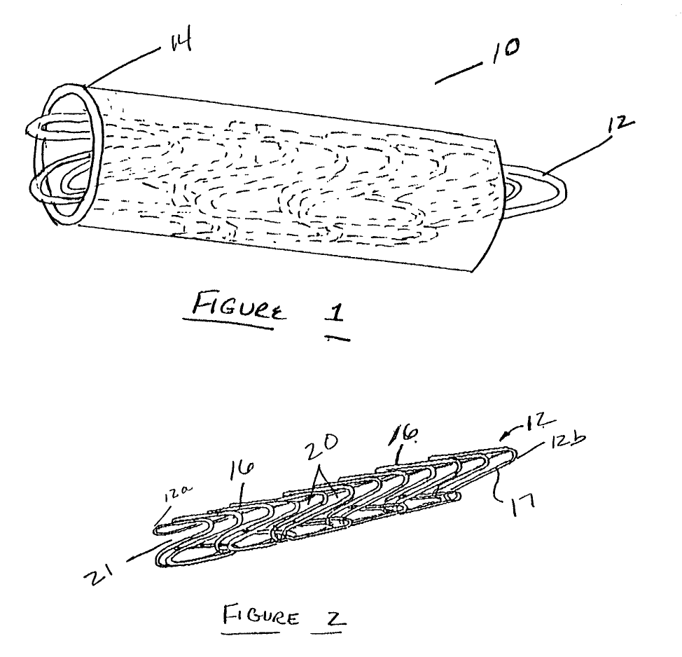

Now referring to the figures, where like elements are identically numbered, FIG. 1 shows a preferred embodiment of a tubular stent-graft prosthesis 10 of the present invention. Prosthesis 10 includes a tubular radially expandable stent 12 having a polymeric sheath 14 on at least an exterior surface thereof. Sheath 14 includes a thin-walled material, preferably having a thickness between 0.005″-0.006″, inclusive. Sheath 14 is made from a film, sheet or tube of polyolefin material such as polyethylene or polypropylene which is more biocompatible with vascular tissue. Polyolefin material is selected because the solubility factor of polyolefins (7.9-8.1) exhibit a more “bio-friendly” reaction with native vessels versus that experienced with conventional materials such as PET polyester and nylon. Those currently utilized materials exhibit a high solubility factor (10.7-13.6) resulting in an exacerbated inflammatory response in lumen tissue which in turn inhibits the effect of therapeutic substances placed thereon.

The polyolefin material that is used in the device may have any of a variety of textures and finishes which promote endothelialization. Such finishes includes smooth finishes that facilitate laminar bloodflow and mesh-like material having improved porosity so as to promote endothelial lining/tissue growth.

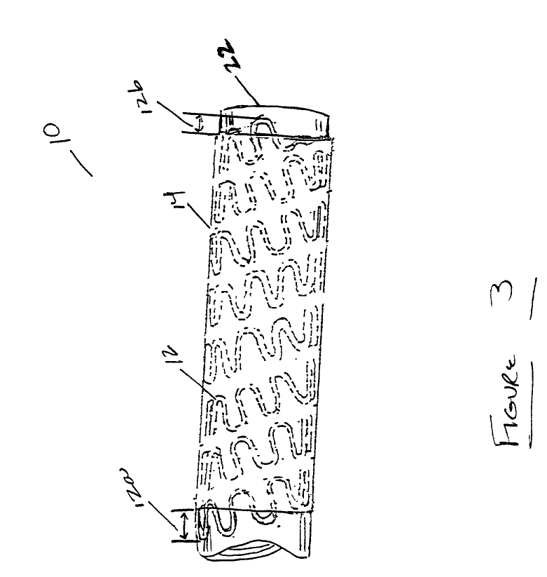

Although a wide variety of stents may be used, FIG. 2 shows a perspective view of one particular stent which may be employed in prosthesis 10. The particular stent shown in FIG. 2 is more fully described in commonly assigned U.S. Pat. No. 5,575,816 to Rudnick, et al. Stent 12 is an intraluminally implantable stent formed of helically wound wire. Multiple windings 16 of a single metallic wire 17, preferably composed of a temperature-sensitive material such as Nitinol, provide stent 12 with a generally elongate tubular configuration which is radially expandable after implantation in a body vessel. The multiple windings 16 of stent 12 define open spaces 20 throughout the tubular configuration and define a central open passage 21 therethrough between opposing extremities 12a and 12b. The helically wound wire configuration not only ensures patency and flexibility, but the open spaces also allow adhesion of tubular layers therethrough.

Although this particular stent construction is shown and described with reference to the present invention, various stent types and stent constructions may be employed in the present invention for the use anticipated herein. Among the various stents useful include, without limitation, self-expanding stent and balloon expandable stents. The stents may be capable of radially contracting as well, and in this sense can be best described as radially distensible or deformable. Self-expanding stents include those that have a spring-like action which causes the stent to radially expand or stents which expand due to the memory properties of the stent material for a particular configuration at a certain temperature. Other materials are of course contemplated, such as stainless steel, platinum, gold, titanium and other biocompatible materials, as well as polymeric stents.

The configuration of the stent may also be chosen from a host of geometries. For example, wire stents can be fastened in a continuous helical pattern, with or without wave-like forms or zig-zags in the wire, to form a radially deformable stent. Individual rings or circular members can be linked together such as by struts, sutures, or interlacing or locking of the rings to form a tubular stent. Tubular stents useful in the present invention also include those formed by etching or cutting a pattern from a tube. Such stents are often referred to as slotted stents. Furthermore, stents may be formed by etching a pattern into a material or mold and depositing stent material in the pattern, such as by chemical vapor deposition or the like.

The fabrication of a composite device of the type shown in FIG. 1 can now be described.

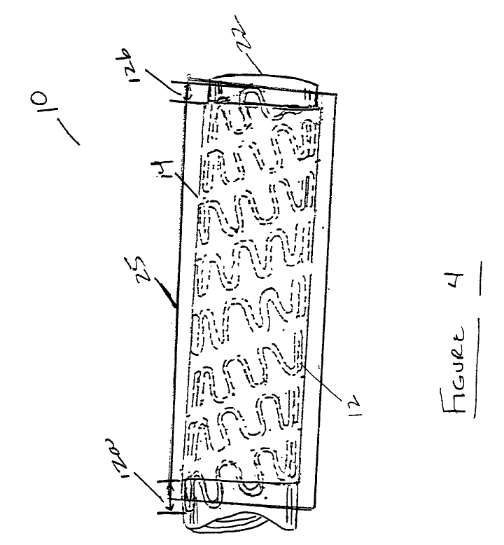

Prosthesis 10 is formed by providing a stent 12 on a mandrel 22 as shown in FIG. 3. A polyolefin sheath or film 14 is wrapped circumferentially around stent 12, as shown in FIG. 4.

As further shown in FIG. 4, a heat shrink tubing 25 is layered over the polyolefin-covered stent. Mandrel 22, carrying stent 12 and sheath 14 thereon, is placed in an oven at 300-400° F. for approximately 10 minutes, or for a time sufficient for sheath 14 to melt enough to become inextricably combined with stent 12. When sufficient melting has been realized, mandrel 22 and newly covered stent 12 are removed from the oven and cooled, allowing the polyolefin material time to cure. Heat shrink tubing 25 is then removed to reveal the finished prosthesis as shown in FIG. 5.

Now referring to FIG. 5, stent 12 and sheath 14 are concurrently removed from mandrel 22 to reveal newly fabricated prosthesis 10. Sheath 14 may be adapted to entirely envelop the stent's exterior surface or leave portions thereof exposed, such as extremities 12a and 12b illustrated in FIG. 5. Such placement of the sheath may be desirable in certain applications where stent exposure assists with anchoring of the stent graft device in a conduit to be treated.

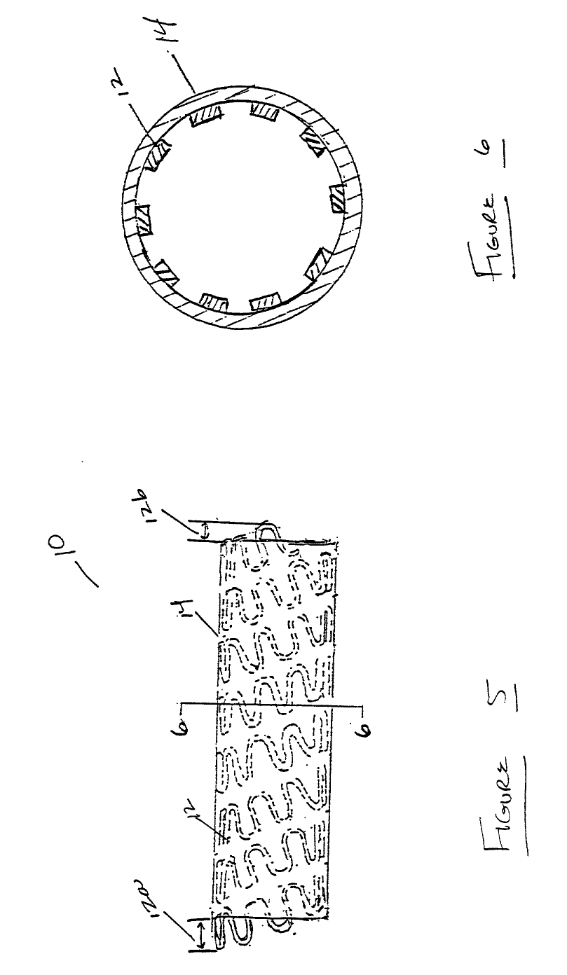

As is evident from FIG. 6, a cross section of prosthesis 10 reveals that sheath 14 circumferentially envelops the outer periphery of stent 12. The covering material can either be flush with the ends of the stent or centered mid-stent allowing approximately 2-3 mm of open stent on both the proximal and distal ends thereof. Upon melting of the polyolefin material, portions of sheath 14 may fill the interstices between adjacent stent windings so as to partially envelope said windings therein. Although sheath 14 appears as a substantially complete tube that is slid over the stent while on the mandrel 22, it is evident that the sheath may be a film or sheet having its opposing edges overlapped and secured to one another to form a tubular structure.

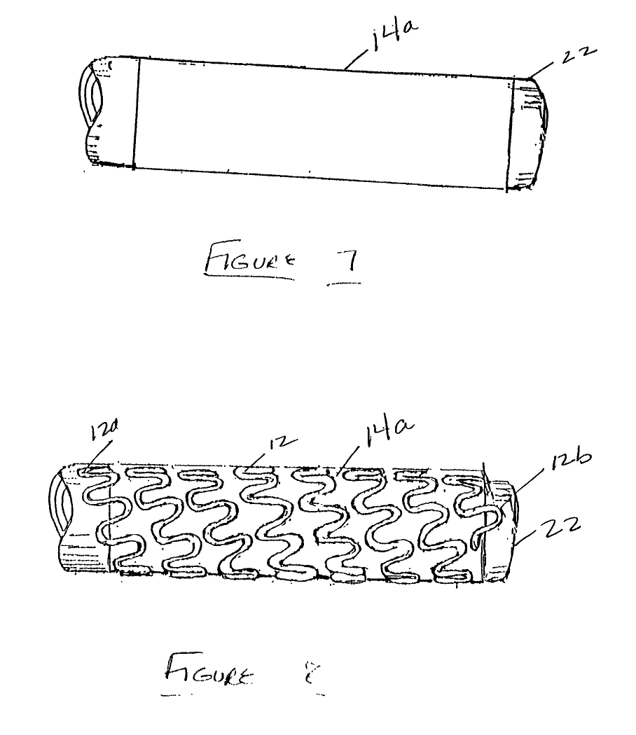

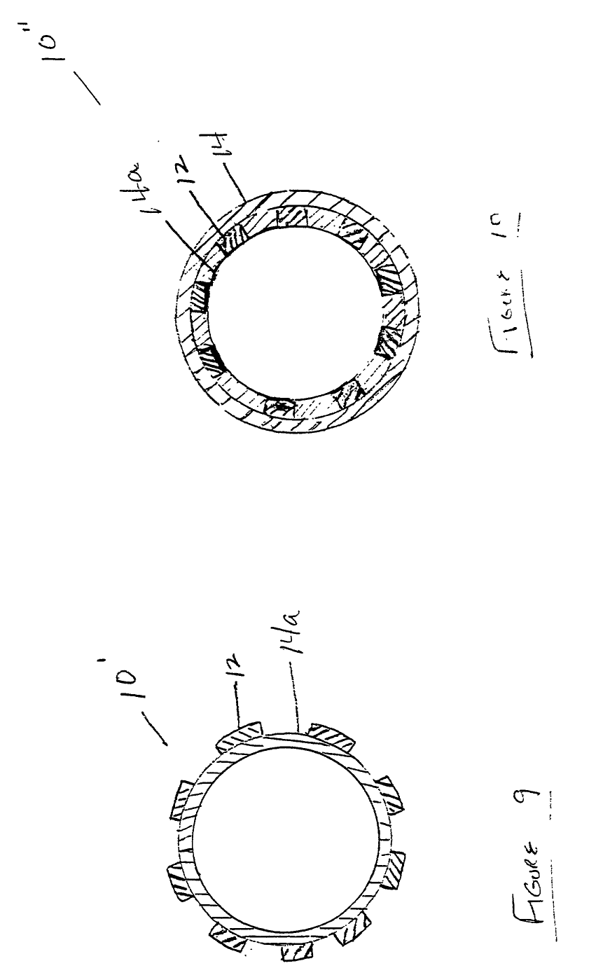

In another embodiment of the present invention, a luminal covering is similarly formed by placing a second sheath 14a of polyolefin material directly on mandrel 22. Sheath 14a is secured to the mandrel prior to affixing stent 12 thereon, as shown in FIG. 7. As further shown in FIG. 8, stent 12 is thereafter placed overlying sheath 14a. After heating of the mandrel as described hereinabove, the sheath and mandrel combination may be removed from the mandrel to produce a prosthesis 10′ having a luminal polyolefin layer disposed circumferentially on a luminal surface of stent 12, as shown in FIG. 9. Similar to the embodiment shown in FIG. 6, sheath 14a may melt so that the polyolefinic material flows into the interstices between adjacent windings, thereby at least partially enveloping said windings therein.

In an additional embodiment of the present invention, both luminal and external layers may be provided by combining the procedures described hereinabove. As shown in FIGS. 7 and 8, respectively, a sheath 14a is first placed on mandrel 22 after which stent 12 is laid thereon. As further shown in FIG. 4, sheath 14 is subsequently disposed about an exterior surface of stent 12. Heat shrink tube 25 is placed over the entire combination and subsequently heated to the requisite temperature. As shown in FIG. 10, prosthesis 10″ is produced which includes a pair of impermeable polyolefin layers having a stent 12 therebetween. Sheaths 14 and 14a may substantially melt into one another along a seam so as to render the two sheaths indistinguishable from one another.

Either or both of the luminal and exterior sheaths 14 and 14a may be provided with an adhesive thereon which permits adherence of the polyolefin structures to one another through the stent openings and simultaneously allows adherence of stent 12 to either or both of the polyolefin structures. The adhesive may be a thermoplastic adhesive and more preferably, a thermoplastic fluoropolymer adhesive such as FEP. A suitable adhesive provides a substantially sealed tube without significantly reducing longitudinal and axial compliance. Alternatively, the two coverings may be affixed by heating them above the melt point of the polyolefin adequately to cause them to thermally adhere.

Polymeric fibers or films can also be attached to stent platforms by suturing the material to the stent. As discussed hereinabove, the covering material can either be flush with the ends of the stent or centered mid-stent allowing 2-3 mm of open stent on both the proximal and distal ends of the stent. To suture the polymeric fiber or film to the stent, the preferred method is to use silk sutures and attach the preferred polyolefin material to the stent at its distal and proximal ends. The number of silk sutures that will hold the tubular polyolefin material to the stent will depend on the stent diameter. Although silk is the preferred suture material, other polymeric materials may be selected from the group consisting of absorbable (i.e., catgut, reconstituted collagen, polyglycolic acid) and nonabsorbable (i.e., silk, cotton and linen, polyester, polyamide, polypropylene and carbon fiber) materials. External factors that govern the selection of suture material include tissue type, temperature, pH, enzymes, lipids and bacteria.

The present prosthetic materials can also be implemented in an implantable vascular prosthesis or graft. “Vascular graft” can mean conventional and novel artificial grafts made of this material constructed in any shape including straight, tapered or bifurcated and which may or may not be reinforced with rings, spirals or other reinforcements and which may or may not have one or more expandable stents incorporated into the graft at one or both ends or along its length. The vascular graft of choice may be introduced into the vessel in any suitable way including, but not limited to, use of a dilator/sheath, placement of the graft upon a mandrel shaft and/or use of a long-nose forceps. The distal ends of the tubular graft and the mandrel shaft may be temporarily sutured together, or the distal end of the vascular graft may be sutured together over the mandrel to accommodate unitary displacement into a vessel, for example, through a sheath after the dilator has been removed. One or both ends of the vascular graft may be sutured or surgically stapled in position on the treated vessel to prevent undesired displacement or partial or complete collapse under vascular pressure.

Where the graft is expandable and in tubular or sleeve form, the diametrical size of the graft may be enlarged in contiguous relationship with the inside vascular surface via a balloon catheter. The tubular graft itself may comprise a biologically inert or biologically active anti-stenotic coating applied directly to the treated area of the remaining vascular inner surface to define a lumen of sufficient blood flow capacity. The graft, once correctly positioned and contiguous with the interior vascular wall, is usually inherently secure against inadvertent migration within the vessel due to friction and infiltration of weeping liquid accumulating on the inside artery wall. The length of the vascular graft preferably spans beyond the treated region of the vessel.

It is anticipated that the covered stent device of the present invention can be coated with hydrophilic or drug delivery-type coatings which facilitate long-term healing of diseased vessels. The polymeric material is preferably bioabsorbable, and is preferably loaded or coated with a therapeutic agent or drug, including, but not limited to, antiplatelets, antithrombins, cytostatic and antiproliferative agents, for example, to reduce or prevent restenosis in the vessel being treated. The therapeutic agent or drug is preferably selected from the group of therapeutic agents or drugs consisting of sodium heparin, low molecular weight heparin, hirudin, prostacyclin and prostacyclin analogues, dextran, glycoprotein IIb/IIIa platelet membrane receptor antibody, recombinant hirudin, thrombin inhibitor, calcium channel blockers, colchicine, fibroblast growth factor antagonists, fish oil, omega 3-fatty acid, histamine antagonists, HMG-CoA reductase inhibitor, methotrexate, monoclonal antibodies, nitroprusside, phosphodiesterase inhibitors, prostaglandin inhibitor, seramin, serotonin blockers, steroids, thioprotease inhibitors, triazolopyrimidine and other PDGF antagonists, alpha-interferon and genetically engineered epithelial cells, and combinations thereof. While the foregoing therapeutic agents have been used to prevent or treat restenosis and thrombosis, they are provided by way of example and are not meant to be limiting, as other therapeutic drugs may be developed which are equally applicable for use with the present invention.

Various changes and modifications can be made to the present invention. It is intended that all such changes and modifications come within the scope of the invention as set forth in the following claims.

What is claimed is:

1. An implantable stent-graft prosthesis for minimizing tissue inflammatory responses, comprising:
an elongate radially adjustable stent having a substantially tubular configuration defining a central open passage therethrough, said stent having proximal and distal extremities and opposed interior and exterior stent surfaces wherein said stent includes plural open spaces extending between said opposed luminal and exterior surfaces so as to permit said radial adjustability;
at least one polymeric tubular structure having a stent contacting surface disposed circumferentially about one of said luminal or exterior stent surfaces;
wherein said polymeric structure is made of a polyolefin material having a softening temperature in the range 300-400° C., inclusive.

2. The stent-graft device of claim 1 wherein said polyolefin material is selected from the group consisting of polyethylene and polypropylene.

3. The stent-graft device of claim 1 wherein said polymeric tubular structure is softened on said stent.

4. The stent-graft device of claim 3 wherein said softened structure melts into said plural open spaces.

5. The stent-graft device of claim 1 further comprising a second polymeric tubular structure having a stent contacting surface disposed circumferentially about the other of said luminal or exterior stent surfaces.

6. The stent-graft device of claim 5 wherein said polymeric tubular structures are secured to one another through said open stent spaces.

7. The stent-graft device of claim 6 wherein said polymeric tubular structures are laminated together through said open stent spaces.

8. The stent-graft device of claim 6 wherein said polymeric tubular structures are adheringly secured through said open spaces of said stent.

9. The stent-graft device of claim 5, wherein at least one of said polymeric tubular structures is formed from an extruded tube.

10. The stent-graft device of claim 5, wherein at least one of said polymeric tubular structures is formed from a seamless sheet having opposed longitudinal edges and wherein said edges are joined to form a tubular structure.

11. A method of manufacturing a stent-graft composite intraluminal prosthetic device, comprising the steps of:
providing an elongate radially adjustable tubular stent, defining opposed luminal and exterior stent surfaces;
placing said stent about a correspondingly sized and shaped mandrel;
placing a polymeric tubular structure circumferentially about at least one of said luminal and exterior stent surfaces so as to contact a stent surface thereadjacent;
heating said stent and said tubular structure for a time sufficient to melt said polymeric structure over said stent; and
removing said stent from said mandrel;
wherein said polymeric structure is made of a polyolefin material having softening temperature in the range 300-400° C., inclusive.

12. The method of claim 11 wherein said polymeric material is selected from the group consisting of polyethylene and polypropylene.

13. The method of claim 11 wherein said melted polymeric structure melts into plural open spaces of said stent.

14. The method of claim 11 further including the step of covering said mandrel with said polymeric material prior to placing said stent thereon.

15. The method of claim 14 further including the step of securing said polymeric structure through said open spaces.

16. The method of claim 15 wherein said securing step includes laminating said polymeric material through said open stent spaces.

17. The method of claim 15 wherein said securing step includes adhering said polymeric tubular structures to one another.

18. An implantable tubular prosthesis that minimizes tissue inflammatory responses, comprising:
an expandable polymeric tubular structure comprising a polyolefin material having a softening temperature in the range of 300° C.-400° C., inclusive, said tubular structure including a tissue contacting outer surface circumferentially defined therearound and an inner blood contacting surface concentric thereto.

19. The implantable tubular prosthesis of claim 18 wherein said polyolefin material is selected from the group consisting of polyethylene and polypropylene.

20. The implantable tubular prosthesis of claim 18 wherein said prosthesis includes a second polymeric tubular structure disposed circumferentially about either of said tissue contacting outer surface and said inner blood contacting surface.

21. The implantable tubular prosthesis of claim 20 wherein said polymeric tubular structures are securable to one another.

22. The implantable tubular prosthesis of claim 21 wherein said polymeric tubular structures are laminated together.

23. The implantable tubular prosthesis of claim 21 wherein said polymeric tubular structures are adheringly secured to one another.

24. The implantable tubular prosthesis of claim 20 wherein said material is extrudable to form said polymeric tubular structures.

25. The graft material of claim 20, wherein at least one of said polymeric tubular structures is formed from a seamless sheet having opposed longitudinal edges and wherein said edges are joined to form a tubular structure.
